(19) United States
(12) Patent Application Publication
(10) Pub. No.: US 2021/0228145 A1
(43) Pub. Date: Jul. 29, 2021

(54) SKIN REACTION TEST PATCH FOR DIAGNOSING DIETARY ALLERGY IN ANIMALS

(71) Applicant: Hye Hyeon HAN, Daejeon (KR)

(72) Inventor: Hye Hyeon HAN, Daejeon (KR)

(21) Appl. No.: 17/033,966

(22) Filed: Sep. 28, 2020

(30) Foreign Application Priority Data
Jan. 23, 2020 (KR) 10-2020-0009248

(51) Int. Cl. A61B 5/00; A61D 7/00

(52) U.S. Cl. CPC A61B 5/411; A61B 5/685; A61B 2503/40; A61B 5/6833; A61D 7/00

(57) ABSTRACT

The present invention relates to a skin reaction test patch for diagnosing a dietary allergy in animals, which easily diagnoses an allergy through a skin reaction test by conveniently and effectively injecting an allergen into the dermal layer of an individual animal, and a method for diagnosing a dietary allergy using the same, and according to the present invention, the dietary allergy of a companion animal can be simply diagnosed within 30 minutes even at home by attaching a band-type patch to the skin surface of the animal and peeling off the patch.

### CROSS-REFERENCE TO RELATED APPLICATION

This application claims priority to and the benefit of Korean Patent Application No. 10-2020-0009248 filed on Jan. 23, 2020, the disclosure of which is incorporated herein by reference in its entirety.

### BACKGROUND

### 1. Field of the Invention

The present invention relates to a skin reaction test patch for diagnosing a dietary allergy in animals, and more specifically, to a skin reaction test patch for diagnosing a dietary allergy in animals, which easily diagnoses an allergy through a skin reaction test by conveniently and effectively injecting an allergen into the dermal layer of an individual animal, and a method for diagnosing a dietary allergy using the same.

### 2. Discussion of Related Art

A dietary allergy indicates that an animal having an antigen for a specific food exhibits an abnormal reaction to a food or food additive. In general, diet-related side effects occur within minutes after exposure, but delayed reactions may occur over hours or even days.

Dietary allergic symptoms are very similar to atopic dermatitis, and thus cannot be diagnosed with the naked eye. There may be symptoms such as non-seasonal pruritus accompanied by digestive problems and secondary skin lesions (papules, erythroderma, excoriation, hyperpigmentation), and symptoms usually appear in the thin skin parts such as feet, ears, face, inguinal area, and perianal area. Specifically, it may be accompanied by symptoms such as diarrhea, vomiting, and earwax. In particular, it is important to distinguish a dietary allergy from atopic dermatitis among various skin diseases because the symptoms are very similar to atopy.

As the most accurate method, a method of finding a specific antigen is the most effective, but the method has a disadvantage of being time consuming. More conveniently, there is a diet-inducing test which distinguishes what food animals are allergic to, but this method has the hassle of having to select homemade food or foods which are manufactured and sold (hydrolyzed feed) and check whether the skin symptoms are improved by restricting the diet for about 8 to 12 weeks. Accordingly, a skin reaction test capable of searching for the causative antigen (allergen) by a non-invasive method in a short time has been widely used.

Specifically, the allergic skin reaction test is a test in which a material that may cause allergic symptoms is applied to the skin and then the extent of reaction is tested to determine the presence or absence of allergies. An allergy refers to a hypersensitivity reaction caused by immunological mechanisms, and is mediated by antibodies or cells. A subject sensitized to allergy-causing materials (allergens) produces specific immunoglobulin E (IgE) antibodies that react with the allergens in the body. This specific antibody is bound to the surface of mast cells that are spread throughout the body, but when an allergen enters through the skin during a skin test, the antibody binds to the allergen to secrete various chemical media such as histamine from the mast cells, and the various chemical media secreted cause an immediate wheal and erythema reaction due to actions such as vasodilation and increased permeability. This reaction peaks around 15 to 20 minutes after the test is performed, and in this case, the size of wheal and erythema is measured with a ruler and when the size of the wheal is 3 mm or more, it may be considered to be a sensitized state.

Therefore, the present invention suggests a skin reaction test patch for diagnosing a dietary allergy, which is capable of simply determining the presence or absence of a dietary allergy even at home by manufacturing a patch to which the allergic reaction test principle is applied and adhering the patch to the skin of a companion animal.

### PRIOR ART DOCUMENT

### Patent Document

(Patent 1) Korean Patent No. 10-1878414

### SUMMARY OF THE INVENTION

The present invention has been to solve the aforementioned problems in the related art, and an object thereof is to provide a skin reaction test patch for diagnosing a dietary allergy in animals in the form of a patch provided with microneedles including a dietary allergen used for a skin reaction test and a biodegradable polymer, PVP.

An object of the present invention is also to provide a method for diagnosing a dietary allergy in animals using the skin reaction test patch for diagnosing a dietary allergy in animals.

To achieve the objects as described above, the present invention provides a skin reaction test patch for diagnosing a dietary allergy in animals, the patch including:

a plurality of microneedles formed by mixing polyvinylpyrrolidone (PVP) and an allergen; and

a pad supporting the plurality of microneedles,

in which the allergen is injected into an individual by the biodegradation of the microneedles.

The skin reaction test patch for diagnosing a dietary allergy in animals according to the present invention is characterized in that 20 types of allergies can be diagnosed at one time by containing various types of allergens in the plurality of microneedles.

The skin reaction test patch for diagnosing a dietary allergy in animals according to the present invention is characterized in that the type of allergen is capable of being positionally identified by containing only a dye in any one or more of the plurality of microneedles.

The skin reaction test patch for diagnosing a dietary allergy in animals according to the present invention is characterized in that the type of allergen is capable of being positionally identified by containing a dye and an allergen in any one or more of the plurality of microneedles.

In the skin reaction test patch for diagnosing a dietary allergy in animals according to the present invention, a height of the microneedles is 100 to 200 μm.

In the skin reaction test patch for diagnosing a dietary allergy in animals according to the present invention, the pad further includes an intermediate layer formed of PVP and a surface layer formed by coating the intermediate layer with a flavoring agent for preventing animal licking.

Further, the present invention provides a method for diagnosing a dietary allergy in animals, which can diagnose 20 types of allergies at one time within 30 minutes by adhering the skin reaction test patch for diagnosing a dietary allergy in animals to the skin surface of an animal and peeling off the patch to inject an allergen into the skin surface of the animal through a plurality of microneedles formed on the patch and distinguishing the regions of the needles through staining to enable the identification of the type of allergen.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1 to 3 are schematic views illustrating a skin reaction test patch for diagnosing a dietary allergy in animals according to an exemplary embodiment of the present invention.

### DETAILED DESCRIPTION OF EXEMPLARY EMBODIMENTS

Hereinafter, the present invention will be described in detail.

The present invention relates to a skin reaction test patch for diagnosing a dietary allergy in animals, and more specifically, to a skin reaction test patch for diagnosing a dietary allergy in animals, which easily diagnoses an allergy by conveniently and effectively injecting an allergen into the dermal layer of an individual animal, and a method for diagnosing a dietary allergy using the same.

According to a skin reaction test patch for diagnosing an allergy proposed by the present invention, the dietary allergy of a companion animal may be simply diagnosed within 30 minutes even at home, 20 types of allergies may be diagnosed at one time by distinguishing the regions of needles through staining, and costs may be reduced by reducing the squandering of instruments and reagents used for diagnosis.

Hereinafter, the benefits and features of the present application, and the methods of achieving the benefits and features will become apparent with reference to Examples to be described below in detail along with the accompanying drawings. However, the present invention is not limited to the Examples to be disclosed below and may be implemented in various other forms, and the present Examples are only provided for rendering the disclosure of the present invention complete and for fully representing the scope of the invention to a person with ordinary skill in the technical field to which the present invention pertains, and the present invention will be defined only by the scope of the claims.

Since the shapes, sizes, ratios, angles, numbers and the like disclosed in the drawings for describing the embodiments of the present invention are illustrative, the present invention is not limited thereto. Further, when it is determined that the detailed description of the related publicly known art in describing the present invention may obscure the gist of the present invention, the detailed description thereof will be omitted.

When ‘include’, ‘have’, ‘consist of’, and the like referred to herein are used, other parts may be added unless “only” is used. When a component is expressed in the singular form, cases involving a plurality of components are included unless otherwise specified. In analyzing the components, it is to be understood that the error range is included even though there is no separate explicit description.

Throughout the specification, like reference numerals indicate like components.

The size and thickness of each component in the drawings are shown for convenience of description, and the present invention is not necessarily limited to the size and thickness of the illustrated components.

Each feature of the various embodiments of the present invention can be partially or wholly coupled to or combined with each other and can be technically variously interlocked and driven to be fully understood by those skilled in the art, and each embodiment may be implemented independently of each other or together in a related relationship.

Meanwhile, as used herein, “allergy” refers to any form of immunological hypersensitivity reaction in which a reaction to a particular allergen may be harmful to the host itself and phenomena caused thereby. Recently, IgE antibody-mediated hypersensitivity reaction type I against allergens present in the external environment of an individual may be narrowly referred to as an allergic reaction, but the allergic reaction in the present invention is not limited thereto, and should be interpreted as an allergic reaction in a broad sense including hypersensitivity reactions (type I-IV) to all allergens present outside and inside the individual.

Therefore, as used herein, “allergen” refers to all types of dietary materials which may induce the above-described immune hypersensitivity reaction in an individual, and may refer to a material which induces an immune hypersensitivity reaction in an individual without being limited to a certain type of external allergen and internal allergen (that is, auto-allergen).

Therefore, as a dietary allergen, the allergen in the present invention may be any one or a combination thereof selected from the group consisting of commonly used dietary materials (feed) or a combination thereof. Hereinafter, for convenience of explanation, the allergen will be described as one of 20 representative feeds, but it should be noted that the allergen of the present invention is not limited thereto.

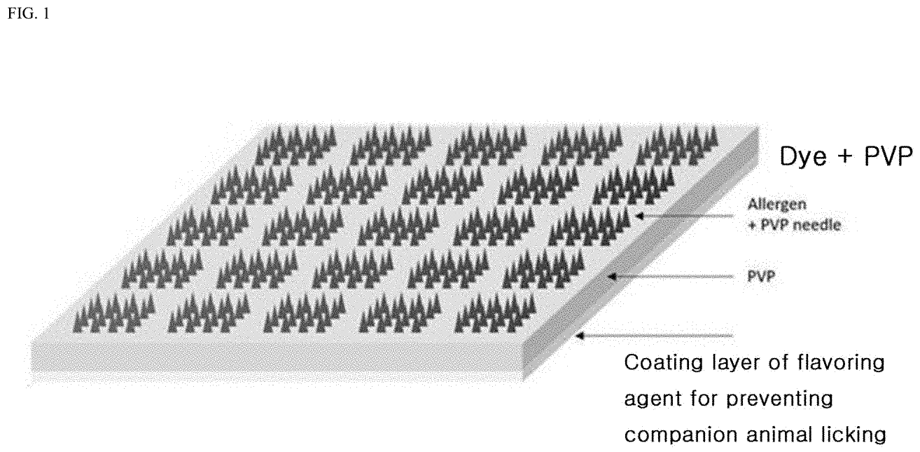

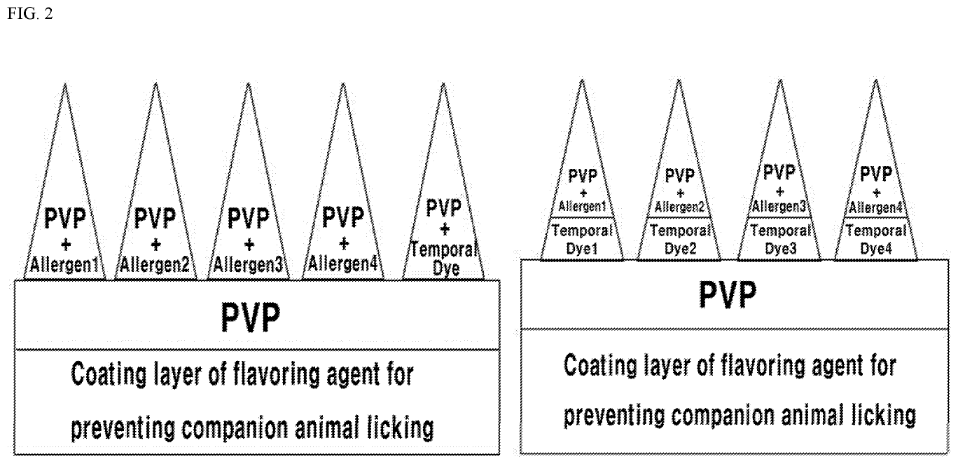

Hereinafter, the microneedle patch of the present invention will be described with reference to accompanying FIGS. 1 and 2.

FIG. 1 is a schematic view illustrating a skin reaction test patch according to various embodiments of the present invention, and FIG. 2 is a plan view illustrating various forms of the biodegradable microneedle patches of the present invention.

Referring to FIG. 1, the skin reaction test patch according to the present invention includes a plurality of microneedles formed in order to inject an allergen into an individual, a dietary allergen contained in the microneedles, and a pad supporting the plurality of microneedles.

In addition, the skin reaction test patch according to various embodiments further includes an intermediate layer formed of a biodegradable polymer on the pad and a surface layer formed by applying a flavoring agent for preventing animal licking on the intermediate layer.

Referring to FIG. 2, a plurality of microneedles made of biodegradable polymers may be formed according to various modified embodiments.

As an example of the present invention, the plurality of microneedles contain different allergens, and include biodegradable materials which can be decomposed by body fluids, enzymes, microorganisms and the like in a living body. Here, biodegradation is interpreted to include both an allergen being injected into a living body and dissolved in a body fluid and an allergen being biodegraded by an enzyme and the like in the body.

In this case, it is preferred that the microneedles are formed of a biodegradable material capable of minimizing side effects at the time of insertion into an individual and effectively delivering a drug. Such a material may be, preferably, biodegradable polymer polyvinylpyrrolidone (PVP). Among various biodegradable materials, the above-mentioned polyvinylpyrrolidone has a property of being degraded in the skin in about 5 minutes, and thus is suitable for application to microneedles for rapid diagnosis. In the related art, among biodegradable materials, hyaluronic acid was usually used, but in the case of hyaluronic acid, it takes several hours to be degraded in the skin, so that it takes a long time for an allergen to be delivered to the skin and cause an allergic reaction. However, polyvinylpyrrolidone is hygroscopic and thus is degraded in the skin in about 5 minutes, so polyvinylpyrrolidone is suitable as a microneedle material for diagnosing a skin reaction by attaching a patch to the skin.

In an exemplary embodiment of the present invention, the plurality of microneedles can respectively contain 20 types of allergens, and at the time of diagnosis using one patch, 20 types of allergies can be diagnosed at one time.

Furthermore, the type of allergen may be positionally identified through transient staining of the microneedles by containing a dye in any one or more of the plurality of microneedles. Specifically, there may be a method capable of indirectly identifying the type of allergen by containing only a dye without containing the allergen in any one or more of the plurality of microneedles, or there may be a method capable of directly identifying the type of allergen by containing the dye and the allergen together, but the method is not limited thereto. Examples of a dye which may be used include rhodamine, phenol red dye, blue dextran dye, Congo red, Malachite green, Rose bengal, and the like, but are not limited thereto.

Meanwhile, the pad may be formed of another known material that is not biodegradable, but preferably may also be formed so as to be biodegraded with microneedles by including the same biodegradable material as the material for the above-described microneedles. Specifically, the intermediate layer of the pad may be formed of polyvinylpyrrolidone among the above-described biodegradable materials, and this is for rapid degradation in the skin when the patch is adhered to the skin of an individual, so that the allergen can be rapidly delivered.

Furthermore, in the pad, a surface layer may be formed by applying a flavoring agent for preventing animal licking on the intermediate layer. The flavoring agent for preventing animal licking is for preventing an animal from licking the patch with its tongue and removing the patch from the skin surface when the patch is adhered to the skin of an individual for diagnosis. Examples of types of flavoring agents which can be used include natural flavoring agents such as sodium citrate-citrus fruit acidity, quinine hydrochloride, quinine-Cornus kousa bark, antipyretic analgesics-bitter taste, diospyrin-astringent taste of persimmon, polyphenols-chemicals found in plants, but are not limited thereto.

Further, when the skin reaction test patch according to various embodiments is adhered to the skin of an animal, an adhesive portion may be further formed on the pad, if necessary, such that the microneedles can be maintained in a fixed state without moving.

When the pad provides supporting force capable of supporting a plurality of microneedles and the skin reaction test patch of the present invention is adhered to the skin of an individual, the shape of the pad is not particularly limited as long as the microneedles can inject an allergen into the individual.

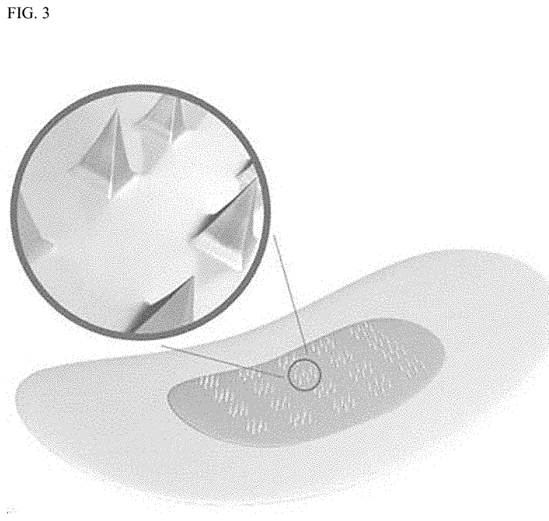

However, as illustrated in FIG. 3, the shape of the pad is preferably a band type which can be conveniently used by a method of adhering the pad to the skin surface of an animal individual and peeling off the pad.

Meanwhile, the microneedles of the skin reaction test patch according to the present invention preferably have a length of about 100 to 150 μm such that the allergen can be injected to a depth of about 100 to 150 mm in the skin of an animal individual. However, this length is exemplary and is not necessarily limited thereto, and it goes without saying that when a point for injecting the allergen is already set, the length of the microneedle is determined so as to be suitable for the point.

According to the skin reaction test patch for diagnosing a dietary allergy in animals according to the present invention, it is possible to simply diagnose the dietary allergy of a companion animal within 30 minutes even at home by adhering a band-type patch to the skin surface of the animal and peeling off the patch. Further, 20 types of allergies can be diagnosed at one time by distinguishing the regions of needles through staining, and costs can be reduced by reducing the squandering of instruments and reagents used for diagnosis.

1. A skin reaction test patch for diagnosing a dietary allergy in animals, the patch consisting of:
a plurality of microneedles, each microneedle formed of a material consisting of biodegradable polyvinylpyrrolidone (PVP), an allergen, and a dye, wherein the dye is selected from the group consisting of rhodamine, phenol red dye, blue dextran dye, congo red, malachite green, and rose bengal; and
a pad supporting the plurality of microneedles and consisting of an intermediate layer formed of PVP and a surface layer formed by coating the intermediate layer with a flavoring agent for preventing animal licking,
wherein the allergen is injected into the animals by biodegradation of the plurality of microneedles,
wherein multiple types of allergies are capable of being diagnosed at one time by containing various types of allergens and dyes in the plurality of microneedles.

2. (canceled)

3. The skin reaction test patch of claim 1, wherein a height of the microneedles is 100 to 200 μm.

4. (canceled)

5. The skin reaction test patch of claim 1, wherein the skin reaction test patch is a band type which is easily adhered to the skin surface.

6. (canceled)
